(19) United States
(12) Patent Application Publication
(10) Pub. No.: US 2011/0166419 A1
(43) Pub. Date: Jul. 7, 2011

(54) ENDOSCOPE HAVING A SHAFT TUBE AND OPTIC

(76) Inventors: Matthias Reif, Philadelphia, PA (US); Thomas Wosnitza, Luneburg (DE)

(21) Appl. No.: 13/001,531

(22) PCT Filed: Jun. 23, 2008
(86) PCT No.: PCT/EP09/04499

§ 371 Date: Mar. 24, 2011

(30) Foreign Application Priority Data
Jun. 28, 2008 (DE) 10-2008-030-130.2

(51) Int. Cl. A61B 1/012

(52) U.S. Cl. 600/154

(57) ABSTRACT

A surgical endoscope having a shaft tube that can be connected at its proximal end area to a flushing water inlet and in the distal end area, has at least one lateral opening and having an optic extending through the shaft tube, said optic being designed with a smaller cross-section than the internal cross-section of the shaft tube, and through said optic an image guide extends along the length thereof, where a suction tube is arranged in the free internal cross-section of the shaft tube next to the optic, the suction opening of said suction tube being arranged distally in front of the shaft tube, said suction tube also being proximally connected to a suction device, and where in the distal end region of the shaft tube a locking element, locking the remaining free internal cross-section, thereof is disposed.

### CROSS-REFERENCE TO RELATED APPLICATION

The present application is based upon and claims the benefit of priority from the PCT/EP2009/004499 filed on Jun. 23, 2009, the entire contents of which are incorporated herein by reference.

### BACKGROUND

1. Field

The present invention generally relates to surgical endoscopes, and particularly to a surgical endoscope having a shaft tube and an optic located within it, with which the area in front of the distal end of the shaft tube can be observed.

2. Description of the Related Art

The use of endoscopes for prostate resection is known. They comprise a blade arranged in the shaft tube, which can cut off tissue in front of the distal end of the shaft tube. Normally this blade is developed as an HF-cutting loop. Because poor visibility prevails during such operations, on account of bleeding, thorough flushing is essential.

Such endoscopes used for resection comprise a continuous flushing system, where the visible area is continuously flushed and from which excess liquid is removed. Flushing is effected through an internal shaft tube and extraction through the annular space between the inner and outer shaft tube, where the outer shaft tube is provided in the distal area with lateral holes, through which extraction takes place.

The resection equipment described is used in conventional transurethral resection, during which relatively small pieces of tissue are cut off, which can be sucked out with the flushing water. From time to time, the insert must be removed from the outer shaft tube, to allow even larger pieces to flow out through the enlarged cross-section.

Recently however, prostrate resection methods are becoming established, such as TUEB (Trans Urethral Enucleation Bipolar) or HOLEP (HOlmium Laser E-nucleation Prostate) where the tissue is cut into largish pieces, which can no longer be extracted through the shaft tube of the endoscope. With such resection operations, large pieces of tissue are found in the bladder, which initially have to be reduced in size there, before they can be passed through the shaft tube to the outside.

For such size reduction purposes morcellators are used, which reduce the size of the tissue and remove it by suction from where its size is reduced. The extracted liquid must be followed up by continuous flushing similar to that used in prostate resection methods.

Similar problems exist with other surgical extraction instruments, such as with simple suction tubes, with which, for example, tissue residues, stone fragments or the like are sucked up from the base of the bladder. Here too, as with the morcellating work described above, we are faced with the problem of having to manoeuvre the suction tube or the suction morcellator with the suction aperture under targeted optic observation and of having to follow up the extracted liquid.

The task of the present invention is to create a device suitable for these purposes.

### SUMMARY

According to the invention, an endoscope with a shaft tube and internal optic is provided, together with a suction tube, e.g. in the form of a suction morcellator, the suction aperture of which is situated in front of the distal end of the shaft tube of the endoscope within the field of observation of the optic. The suction aperture and the distal end of the shaft tube are at a distance from one another. With this design, the morcellator and the suction tube can be very easily controlled and observed and can suck up or morcellate the selected pieces precisely. Because suction is carried by the suction tube or morcellator, the lateral holes in the shaft tube are used to supply liquid to enable continuous flushing, with supply and extraction of liquid by suction in the working area, which results in a clear field of vision. The sealing element blocks the shaft tube in an axial direction, so that liquid cannot escape directly axially from the shaft tube, but only at the side. This results in a flow with good flushing action, but without disturbance from strong axial flow.

The sealing element that seals the shaft tube at the distal end can be provided in any way, for example as an elastic sealing stopper, as a sealing lip or as a suitable seal, which can be arranged on the shaft or suction tube. Advantageously, according to claim 2, the sealing element is, however, arranged in the end area of the optic and can be used with it.

Advantageously, according to claim 3, the optic and the morcellator can be removed from the shaft tube. This facilitates cleaning for reuse and creates opportunities for combination with other instruments.

Advantageously according to claim 4, the shaft tube is formed in such a way that it can also be used as the outer shaft of a resection device mentioned at the outset. This creates the great advantage that the same outer shaft can be used for the resection and for subsequent morcellating. Thus, it can remain in situ in the urethra throughout the operation, therefore reducing the negative impact on the patient from inserting and removing the shaft several times.

### BRIEF DESCRIPTION OF THE DRAWINGS

The invention is shown diagrammatically and by way of example in the drawing.

FIG. 1 shows an axial section through the distal end area of the shaft tube of an endoscope according to the invention:

FIG. 2 shows a side view of the shaft tube,

FIG. 3 shows a side view of an insert for the shaft tube of FIG. 2,

FIG. 4 shows a section along line 4-4 in FIG. 1 and,

FIG. 5 shows a section along line 5-5 in FIG. 1.

### DETAILED DESCRIPTION

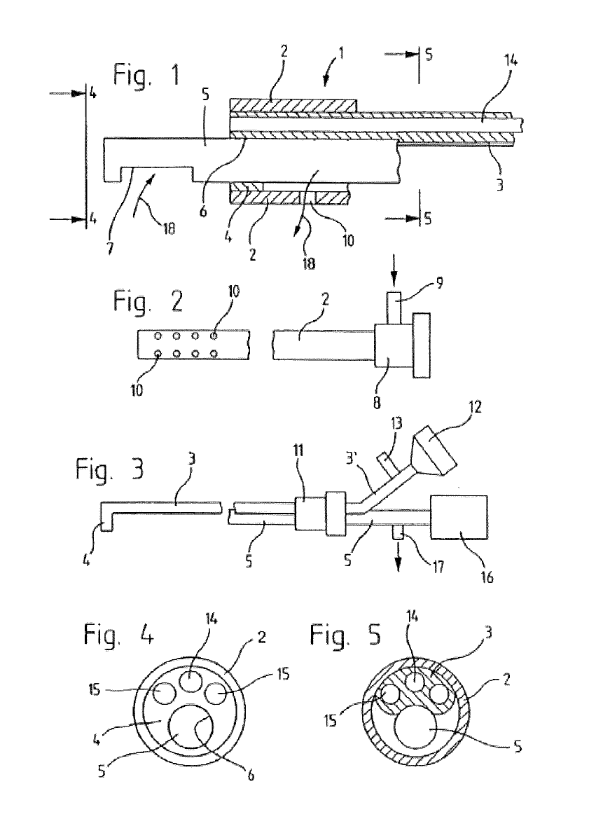

FIG. 1 shows, in longitudinal section, the distal tip of a urological endoscope 1 with a shaft tube 2 forming the outer wall and an optic 3 located therein, which, as FIGS. 1 and 3 show, is formed in the distal end region as a sealing element 4, which seals the internal cross-section of the shaft tube 2. Proximally adjacent to the sealing element 4, however, as FIG. 1 shows along the section line 5-5, the cross-section of the optic 3 is smaller, which is also quite clearly shown in FIG. 5.

In the free internal cross-section of the shaft tube 2 available at this point next to the optic 3, there is space to introduce an elongated, rod shaped morcellator 5, which, as shown in FIGS. 1 and 4, passes through a hole 6 parallel to the axis of the shaft tube 2, in the sealing element 4 and is positioned with its distal end area in front of the distal end of the shaft tube 2. Here, the morcellator 5 has a side suction aperture 7, which is used for morcellating and sucking up the morcellated material.

FIG. 2 shows a side view of the shaft tube 2. At the proximal end area 8 of the shaft tube 2, a flushing water supply connection 9 is attached, with which liquid can be introduced into the inside of the shaft tube 2 in the direction of the arrow. In the distal end area of the shaft tube 2 the latter comprises holes 10 on all sides, of which one can also be seen in FIG. 1.

As FIG. 3 shows, the optic 3 has a coupling 11 in its proximal end area, which is inserted in the proximal end area 8 of the shaft tube 2 and can be coupled there to form a seal. From the coupling 11, the optic continues proximally in a angled piece 3′, at the end of which are arranged an eyepiece 12 and a lateral light guide connection point 13. The eyepiece 12 is located at the proximal end of an image guide 14, which passes through the length of the optic 3. The light guide connection point 13 is located at the proximal end of two parallel light guides 15, which likewise pass through the length of the optic 3 and emit light in its distal end face.

Unlike the explanation in the embodiment, the optic 3 can, for example, be developed as a video optic, where, in the distal end area a video camera is arranged, from which a video cable, forming the image guide, runs in a proximal direction. The light guides may be missing from the optic, as they may, for example, be located separately from the endoscope shown.

As shown in FIG. 3, the morcellator 5 is sealed and passes through the coupling 11 of the optic 3 and protrudes proximally from it. A morcellator drive 16 is connected at its proximal end, together with a side connecting piece 17, from which the liquid from the inside of the morcellator 5 is sucked off.

As a departure from the design depicted, instead of the morcellator 5, a simple suction tube can be used, which, for example, can be designed like the morcellator 5 shown, but without the morcellating function. Other suction instruments may also be used instead of the morcellator 5, such as arthroscopic shavers.

If the morcellator 5, which can be separated from the optic 3 by removing it in the proximal direction, is inserted in the latter according to the FIG. 3 configuration and if the optic 3 is in turn inserted in the shaft tube 2 of FIG. 2, then we obtain, in the distal end area, the configuration shown in FIGS. 1, 4 and 5. The inside of the shaft tube 2 is sealed at its distal end by the end body 4. Flushing liquid introduced by the flushing water feed connection 9 into the shaft tube 2, escapes from the shaft tube 2 through the side holes 10 and is sucked in through the aperture 7 by the morcellator 5. Through said aperture, the rotating blade of the morcellator, which is driven by the drive 16, is accessible from the outside. The liquid supply and discharge is indicated in FIG. 1 by arrows 18.

As already explained, the optic 3 and the morcellator 5 can be removed from the shaft tube 2 in proximal direction. As the diagram in FIG. 2 makes clear, the outer shaft 2 in its proximal end area 8 is developed in such a way that not only the optic 3 with its coupling 11 can be connected to it, but that alternatively, the resection inserts of resection equipment can be attached to the shaft tube 2, such as those, for example, which operate according to the TUEB or HOLEP methods.

If the shaft tube 2 is used as the outer shaft of a resection device, then liquid is usually supplied in an axial direction from the distal end of the shaft, in the distal direction and is sucked out through the side holes in the outer shaft. In the present use of the shaft tube 2, together with a suction tube or a suction morcellator 5, suction is performed by the suction tube or morcellator and liquid is supplied through the holes 10. Under certain circumstances, even one single hole 10 may be sufficient.

As shown by the arrows 18 in FIG. 1, a circular flow is to be achieved between liquid supply and liquid discharge. However, in any event, the sealing element 4 should prevent liquid from flowing in axially. This could wash away pieces of tissue that are to be morcellated and therefore prevent morcellation or discharge, whilst in the case of the design according to the invention as per FIG. 1, the liquid flow drives tissue particles in the direction of the suction aperture 7.

1. A surgical endoscope comprising a shaft tube that can be connected at its proximal end area to a flushing water inlet and in the distal end area, has at least one lateral opening and having an optic extending through the shaft tube, said optic being designed with a smaller cross-section than the internal cross-section of the shaft tube, and through said optic an image guide extends along the length thereof, where a suction tube is arranged in the free internal cross-section of the shaft tube next to the optic, the suction aperture of said suction tube being arranged distally in front of the shaft tube, and connected proximally to a suction device, and where in the distal end area of the shaft tube a sealing element, which seals the remaining free internal cross-section, is located.

2. The surgical endoscope according to claim 1, wherein the distal end area of the optic forms the sealing element.

3. The surgical endoscope according to claim 1, wherein the optic and the suction tube are designed so that they can be removed from the shaft tube.

4. The surgical endoscope according to claim 3, wherein the shaft tube is formed as the outer shaft of a resection device for transurethral prostate resection.
